(19) United States
(12) Patent Application Publication
(10) Pub. No.: US 2023/0096515 A1
(43) Pub. Date: Mar. 30, 2023

(54) HAND STIMULATION DEVICE TO FACILITATE THE INVOCATION OF A MEDITATIVE STATE

(71) Applicant: Hyper Ice, Inc., Irvine, CA (US)

(72) Inventors: Sarah McDevitt, Kensington, CA (US); Brian Bolze, San Francisco, CA (US); Nagaraj Hegde, Noordwijkerhout (NL)

(21) Appl. No.: 17/935,756

(22) Filed: Sep. 27, 2022

Related U.S. Application Data
(60) Provisional application No. 63/248,746, filed on Sep. 27, 2021.
(60) Provisional application No. 63/248,771, filed on Sep. 27, 2021.

(51) Int. Cl. A61M 21/02

(52) U.S. Cl. CPC A61M 21/02; A61M 2021/0072

(57) ABSTRACT

A hand stimulation device that includes a housing defining an interior plenum and having a top portion and a bottom portion; a pair of drive electrodes structured and arranged in a first region of the top portion; a pair of sense electrodes structured and arranged in a second region of the top portion; a processing device disposed within the interior plenum and in communication with each of the pair of drive electrodes and the pair of sense electrodes; at least one sensing device adapted to measure and collect biometric data about the user; and at least one motor that is adapted to generate at least one of tactile feedback and a haptic pattern to a user.

### CROSS-REFERENCE TO RELATED APPLICATIONS

The present application claims priority to U.S. Provisional Patent Application No. 63/248,746 filed Sep. 27, 2021 and U.S. Provisional Patent Application No. 63/248,771 filed Sep. 27, 2021, each of which is hereby incorporated by reference herein in its entirety.

### FIELD OF THE INVENTION

This invention relates generally to a hardware appliance operative with a mobile device executing an application. More particularly, the hardware appliance is in the form of a hand stimulation device to facilitate invocation of a meditative state.

### BACKGROUND OF THE INVENTION

The salutary effects of meditation have been known for centuries. It would be desirable to leverage advances in technology to facilitate the invocation of a meditative state. More particularly, it would be desirable to provide a hardware appliance in the form of a hand stimulation device operative with a mobile device executing an application to facilitate the invocation of a meditative state.

### SUMMARY OF THE INVENTION

In a first aspect, the present invention relates to a hand stimulation device. In some embodiments, the device includes a housing defining an interior plenum and including a top portion and a bottom portion; a pair of drive electrodes structured and arranged in a first region of the top portion; a pair of sense electrodes structured and arranged in a second region of the top portion; a processing device disposed within the interior plenum and in communication with each of the pair of drive electrodes and the pair of sense electrodes; one or more sensing device (e.g., a heart rate sensor, a heart rate variability sensor, a moisture sensor, and a temperature sensor, an electrocardiogram (ECG), and combinations thereof) adapted to measure and collect biometric data about the user; and one or more motor (e.g., a plurality of vibration motors configured an different axes) that is adapted to generate tactile feedback and/or a haptic pattern (e.g., a pattern that syncs with music) to a user.

In some implementations, the pair of drive electrodes and the pair of sense electrodes are structured and arranged to measure a user's respiration rate remotely using bioimpedance. In some variations, the bioimpedance measurement includes a first electrode of the pair of drive electrodes that is structured and arranged: to deliver a current to a first electrode of the pair of sense electrodes in excess of 16 μA at a drive frequencies greater than 32 kHz; to deliver current selected from the group consisting of: 28 μA, 29 μA, 30 μA, 31 μA, 32 μA, 33 μA, 34 μA, 35 μA, or 36 μA, a range from 18-60 μA, a range between 22-52 μA, a range between 26-44 μA, and a range between 30-36 μA; and/or to deliver current at a frequency selected from the group consisting of: 60 kHz, 70 kHz, 80 kHz, 90 kHz, 100 kHz, and a range from 36-128 kHz.

In some applications, each of the first pair of drive electrodes and the second pair of sense electrodes is a dry electrodes and/or a tetra polar electrode. Moreover, each of the first pair of drive electrodes and the second pair of sense electrodes is (i) configured to contact an anatomical region of the user (e.g., the thumbs of the user) remote from a chest of the user and (ii) adapted to capture a change in impedance due to an expansion (inhale) and a contraction (exhale) of the user's lungs.

In some implementations, the sensing device is structured and arranged to transfer sensed data to the processing device and/or remotely. Sensed data may include one or more of: a user's breathing performance, a user's adherence to breathing protocols, and a user's heart rate and the sensed data may be selectively displayable on an input/output device.

In some applications, the device may also include one or more of: a memory that (i) is configured to include a plurality of programs for performing at least one of a plurality of hand stimulation sessions on the device and/or (ii) is structured and arranged to record and store at least one session completed by the user; a device for dampening sound generated by the at least one motor; a heating element; a plurality of light emitting devices that are adapted to display biometric data readings; a form factor capable of being selectively inflated and deflated.

In a second aspect, the present invention relates to a system for performing a meditative, breathing session. In some embodiments, the system includes a hand stimulation device having a housing defining an interior plenum and including: a top portion and a bottom portion, a pair of drive electrodes structured and arranged in a first region of the top portion, a pair of sense electrodes structured and arranged in a second region of the top portion, a processing device disposed within the interior plenum and in communication with each of the pair of drive electrodes and the pair of sense electrodes, one or more sensing device adapted to measure and collect biometric data about the use, and one or more motor that is adapted to generate at least one of tactile feedback and a haptic pattern to a user; a client device in communication with the hand stimulation device; and a server in communication with the client device and the hand stimulation device.

### BRIEF DESCRIPTION OF THE FIGURES

The invention is more fully appreciated in connection with the following detailed description taken in conjunction with the accompanying drawings, in which:

FIG. 1 illustrates a system configured in accordance with an embodiment of the invention.

FIG. 2 illustrates electronic components associated with a hand stimulation device configured in accordance with an embodiment of the invention.

FIG. 3 is a perspective view of a housing for a hand stimulation device configured in accordance with an embodiment of the invention.

FIG. 4 is a front view of a top sphere of a hand stimulation device configured in accordance with an embodiment of the invention.

FIG. 5 illustrates a user engaging a hand stimulation device configured in accordance with an embodiment of the invention.

FIG. 6 illustrates a breathing session executed by the hand stimulation device in accordance with an embodiment of the invention.

FIG. 7 illustrates a soothing session executed by the hand stimulation device in accordance with an embodiment of the invention.

FIG. 8 illustrates a wandering thoughts session executed by the hand stimulation device in accordance with an embodiment of the invention.

FIG. 9 illustrates an interval bell session executed by the hand stimulation device in accordance with an embodiment of the invention.

FIG. 10 illustrates an instructor session executed by the hand stimulation device in accordance with an embodiment of the invention.

FIG. 11 illustrates a biofeedback session executed by the hand stimulation device in accordance with an embodiment of the invention.

FIG. 12 illustrates a fade session executed by the hand stimulation device in accordance with an embodiment of the invention.

FIGS. 13A and 13B illustrates an exemplary device in accordance with an embodiment of the invention.

FIG. 14 illustrates an exemplary device having dry electrodes in accordance with an embodiment of the invention.

FIG. 15 shows an exemplary bioimpedance waveform in accordance with an embodiment of the invention.

FIG. 16 shows an illustrative use of the device in a meditation studio/room setting in accordance with an embodiment of the invention.

FIG. 17 shows an illustrative use of the device in a remote setting in accordance with an embodiment of the invention.

Like reference numerals refer to corresponding parts throughout the several views of the drawings.

### DETAILED DESCRIPTION OF THE INVENTION

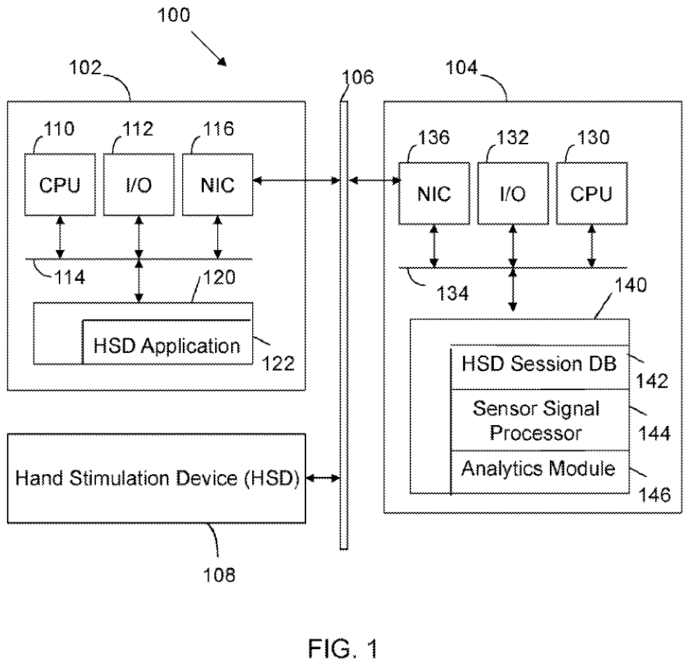

FIG. 1 illustrates a system 100 configured in accordance with a first embodiment of the invention. The system 100 includes a client device 102 connected to a server 104 via a network 106, which may be any combination of wired and/or wireless networks. A hand stimulation device (HSD) 108 is also connected to the network 106.

In some implementations, the client device 102 may include, for the purpose of illustration rather than limitation, a computer, a table, a pad, a mobile device, a wearable device, and the like. In some variations, the client device 102 may include a central processing unit (CPU) 110 and input/output (I/O) devices 112 connected via an internal bus 114. The input/output devices 112 may include, for example, a keyboard, mouse, a touch display, and the like. Optionally, a network interface circuit (NIC) 116 may also be connected to the internal bus 114 to provide connectivity to the network 106. A memory 120 may also be connected to the internal bus 114. Advantageously, the memory 120 stores a hand stimulation device (HSD) application 122 that includes instructions that are executable by the central processing unit 110. The HSD application 122 is structured and arranged to be operative to interact with the HSD 108 via network 106, such as through a WiFi or Bluetooth® connection. The HSD application 122 may also be configured to be operative to communicate with server 104.

In some implementations, server 104 may include a central processing unit (CPU) 130, input/output (I/O) devices 132, an internal bus 134, and a network interface circuit (NIC) 136. A memory 140 may be connected to internal bus 134. Advantageously, the memory 140 is structured and arranged to store instructions executable by the central processing unit 130. In one embodiment, the memory 140 may store an HSD session database 142. The HSD session database 142 is a repository of hand stimulation sessions, where each hand stimulation session includes a sequence of cycles and where each cycle is a sequence of vibration intensity values applied to motor of the HSD 108, resulting in controlled vibration of the HSD 108. For example, in proper use, a user holds the HSD 108 in his or her hand(s). The vibration intensity values are configured to invoke in the user a meditative state. In some implementations, individual sessions of the HSD session database 142 may be downloaded from the HSD database 142 in the server 104 to the client device 102 and/or directly to the HSD 108 via network 106.

In some applications, the server memory 140 may also be configured to store a sensor signal processor 144 that is adapted to collect sensor signals from the HSD 108. Preferably, the sensor signal processor 144 is structured and arranged to evaluate the signals and to selectively provide feedback to the HSD application 122 for consumption by a user of the HSD 108.

The memory 140 may also be adapted to store an analytics module 146. The analytics module 146 may be adapted to include instructions executed by the server central processing unit 130 to supply HSD analytic information, such as, for the purpose of illustration rather than limitation, HSD session participation history, sensor signal analytics, recommended HSD sessions, and the like. In an illustrative embodiment, the HSD analytic information may be conveyed over network 106 to the HSD application 122. An output device 112 (e.g., a display) of the client device 102 may display the conveyed HSD analytic information to a user. The HSD application 122 may also incorporate analytics that are generated and displayed locally.

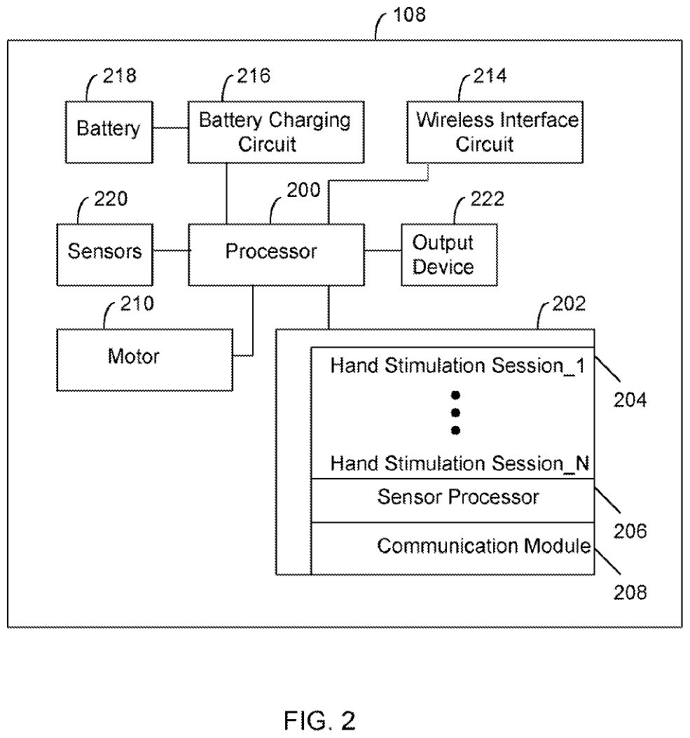

FIG. 2 illustrates an exemplary HSD 108 configured in accordance with one embodiment of the invention. In some implementations, the HSD 108 may include a processor 200 in communication with a memory 202. The memory 202 is structured and arranged to store a set of hand stimulation sessions 204 (e.g., hand stimulation sessions 1 through N), each of which may be executed by the processor 200. Using a (e.g., discrete) hand stimulation session, the processor 200, in turn, is configured to coordinate and control a (e.g., vibratory) motor 210 to apply vibration intensity values to the HSD 108. Each hand stimulation session is configured to guide a user into a meditative state, maintain the user in the meditative state, and then guide the user out of the meditative state. Preferably, each hand stimulation session provides gentle, subtle rhythms, and cues designed to guide and aid individual meditation.

In some applications, the memory 202 may be configured to store a sensor processor 206 that is adapted to collect and process signals transmitted from sensors 220. By way of example, the sensors 220 may be in the form of electrodes, as discussed below, and may include an electrocardiogram (ECG) sensor, a moisture sensor, a temperature sensor, and the like. Such signals may be evaluated to access parameters associated with a meditative state.

The memory 202 may also be structured and arranged to store a communication module 208. In some implementations, the communication module 208 may be configured to coordinate and control communications between the client device 102 and the server 104. More specifically, the communication module 208 may be adapted to access a wireless interface circuit 214 to coordinate such communications. For example, the wireless interface circuit 214 may be structured and arranged to receive HSD sessions from the server HSD session database 142 that are transmitted to the HSD 108 by the server 104 via the network 106. The wireless interface circuit 214 may also be configured to transmit hand stimulation session utilization data, for example, to the client device HSD application 122 and/or to the server HSD session database 142.

The HSD 108 may also include a battery charging circuit 216 and an associated power source (e.g., battery) 218. In some variations, the battery charging circuit 216 may include a physical connector, such as a USB port, or an inductive connection. The HSD 108 may also include an output device 222. In some applications, the output device 222 may be a display or a more basic form of output, such as, for example, a sequence of light emitting diodes (LEDs).

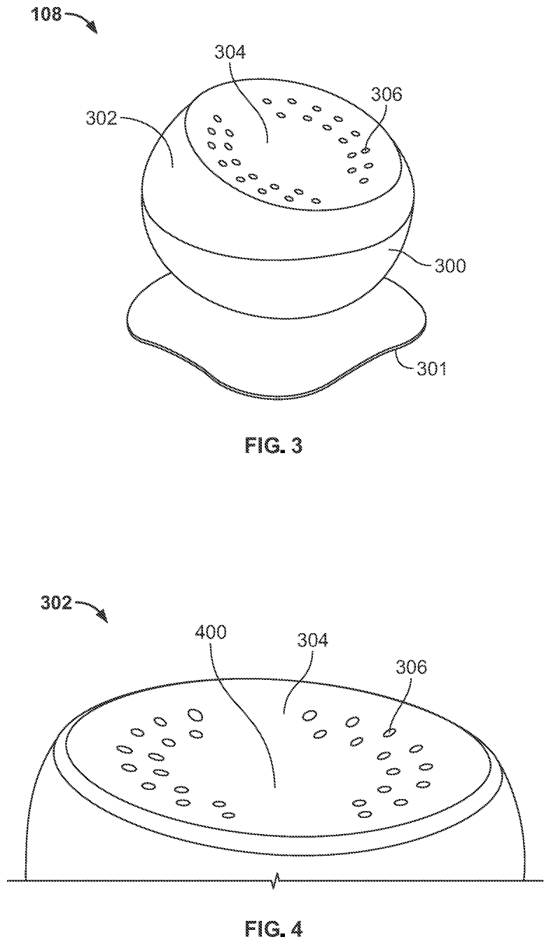

FIG. 3 is a perspective view of an illustrative HSD 108 configured in accordance with one embodiment of the invention. In some implementations, the HSD 108 may include a bottom sphere 300 and a top sphere 302 that are operatively and physically connected to each other. The points of connection of the top 302 and bottom spheres 300 define a central plane. The top sphere 302 and bottom sphere 300 further define an interior volume in which components described above in connection with FIG. 2 may be housed. In one embodiment, the HSD 108 has a diameter of between 80 and 110 mm, preferably approximately 95 mm.

In some applications, the bottom sphere 300 may be shaped like a ball, such as a round ball or an oblong ball. Preferably, the shape of the bottom sphere 300 may be selected to facilitate comfortable engagement with a hand of a user. In some variations, the bottom sphere 300 may have a smooth surface, a dimpled surface, or a textured surface. In one embodiment, the bottom sphere 300 may be formed of wood (e.g., wood with stain and a light varnish finish). The bottom sphere 300 may include a bottom surface that is planar or substantially planar so that the bottom surface may rest on a base 301. The base 301 may include a power charging circuit that is adapted to engage with a battery charging circuit 216. In one embodiment, the planar or substantially planar surface has a diameter of approximately 40 mm.

In some applications, the top sphere 302 may include a concave portion 304 that is formed at an angle (e.g., between 30 and 60 degrees, preferably around 45 degrees) to the central plane. The concave portion 304 is structured and arranged to host a plurality of electrodes 306 that are distributed about the concave portion 304. In some variations, the electrodes 306 may be formed of stainless steel and may be configured to protrude from the surface of the concave portion 304 by approximately 0.5 mm to 3 mm. As shown in FIG. 3, the electrodes 306 may be arranged in a plurality (e.g., two or more) concentric semi-circles on the left and right half of the surface of the concave portion 304. Such an arrangement facilitates engagement of the right and left thumbs of a user with the electrodes 306. The top sphere 302 may be configured to have a smooth surface, a dimpled surface, or a textured surface. In one embodiment, the top sphere 302 may be formed of plastic with a matte finish.

Referring to FIG. 4, the concave portion 304 and electrodes 306 of the top sphere 302 are shown in greater detail. In some embodiments, the top sphere 302 may also include a set of LEDs 400, for example, disposed in the center of the concave portion 304. The LEDs 400 may be used to communicate information, such as session progress, biofeedback results, and diagnostic information, such as charging, low battery, Bluetooth® pairing, and the like. Advantageously, the LEDs 400 are operative as an output device 222 to convey such information as: device on, session progress, and the like. In one embodiment, there may be nine (9) white LEDs and one (1) RGB LED.

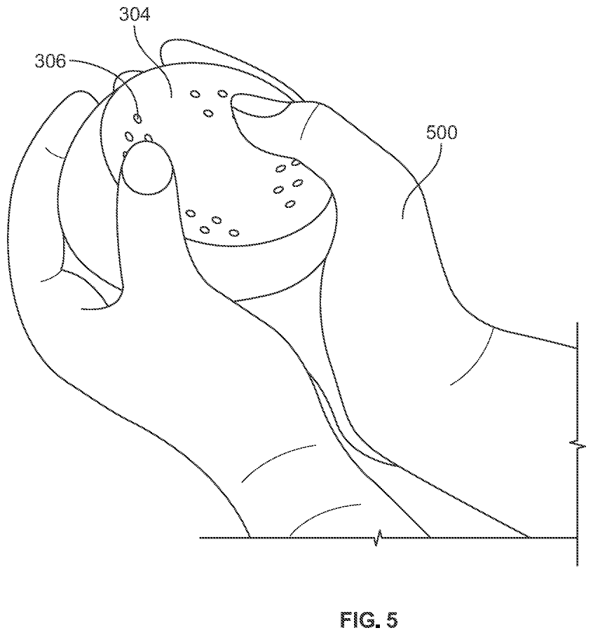

FIG. 5 illustrates a hand 500 of a user engaging the HSD 108. More particularly, the figure illustrates the thumbs of a user engaging the electrodes 306 on the concave portion 304. In one embodiment, the electrodes 306 may be made of stainless steel with a matte finish. In some implementations, the electrodes 306 may be domed so as to protrude 1 mm or less from or above the concave portion 304. The electrodes 306 may be circular, oval, rectangular, or any other polygonal shape. Preferably, the electrodes 306 may be positioned to accommodate users having different hand and finger sizes. Typically, a user's thumbs rest on two or more electrodes 306 in natural reach; however, other fingers may also engage the electrodes 306. Optionally, the HSD 108 may be configured without electrodes 306. In such a case, the vibration intensity values applied to the motor 210 vibrate the entire HSD 108, but the user does not have the additional tactile sensation provided by the electrodes 306.

One or more of the electrodes 306 may be substituted with a sensor 220 of the type earlier described. For example, in one embodiment, the sensor 220 may be structured and arranged to measure electrocardiogram (ECG) signals from a user via contact with the user's thumbs or hands. The ECG data may be used to analyze heart health, heart rate, heart rate variability, respiration rate, and nervous system activity. In particular, the P, Q, R, S, and T waves of ECG activity may be analyzed. These metrics may be used to determine personal heart rate variability baselines, stress and calm thresholds for stressed and calm states, and indicators related to additional health goals, such as fitness and risk of over-training, risk of fatigue, smoking cessation, weight loss, stress management, anxiety, mental health, and the like.

In some implementations, the ECG data may be processed by one or more of the sensor processor 206 of HSD 108, the HSD application 122 on the client device 102, and the sensor signal processor 144 on the server 104. The sensor data may be used to alter vibration intensity values or information conveyed by the output device 222. The sensor signal processor 144 may evaluate sensor signals to derive new HSD sessions, which are loaded into the HSD session database 142. Such sessions may also be downloaded to the HSD application 122, which may convey it to the HSD 108, for example, via a Bluetooth® connection. The sensor signal processor 144 may also generate data for consumption by the server analytics module 146. The HSD application 122 may query the analytics module 146 for various HSD analytical activity. For example, a user may obtain data about a single session, multiple sessions over time, and/or suggested sessions. In one embodiment, the HSD application 122 may be used to play an audio guided meditation track while the HSD 108 is used. The audio track may be obtained, for example, from the HSD session database 142.

Having described a system 100 for hand stimulation for meditation purposes, a variety of hand stimulation sessions that may be utilized on the system 100 will now be described. As previously discussed, a hand stimulation session may include a sequence of cycles, in which each cycle includes a sequence of vibration intensity values applied to the electrodes 306. The vibration intensity values may be characterized as a percentage of the maximum vibration intensity of the HSD motor 210.

In some implementations, hand stimulation sessions 204 may be obtained from the server HSD session database 142. For example, in one embodiment, HSD application 122 of the client device 102 may be configured to communicate with the server 104 via the network 106. More particularly, the HSD application 122 May be adapted to view and select (e.g., discrete) HSD sessions from the server HSD session database 142. The HSD application 122, may then transmit a selected HSD session to the HSD 108, e.g., via the wireless interface circuit 214 of the HSD 108. Optionally, the HSD application 122 may allow a session to be altered for user preference, instructor control, and/or biofeedback.

One type of HSD session is referred to as a breathing cycle session. The breathing cycle session mimics a human breathing rhythm at a rate and pattern that is optimal for the body's homeostatic processes, efficiency of the heart and autonomic nervous system activities, and recovery. For most users, this optimal breathing rate is between 4.5 and 7 breaths per minute (BPM), with the average at 6 BPM. Typically, 40% of a respiration cycle is spent in inhale and 60% of the respiration cycle in exhale. Accordingly, a breathing cycle session may be designed so that the vibration is a breathing guide that aids the user to achieve optimal recovery during a session. The user inhales as the vibration intensity ramps up and exhales as it ramps down. Although the breathing cycle session will be described using a discrete guidance pattern, this is done for the purposes of brevity and illustrative purposes only. Indeed, those of ordinary skill in the art can appreciate that varying vibration intensity to model a breathing cycle that comprises 40% of a respiration cycle is spent in inhale and 60% of the respiration cycle in exhale and about 6 BPM is merely intended to be illustrative. The ratio or inhale-to-exhale, as well as the number of breaths per minute, may be varied.

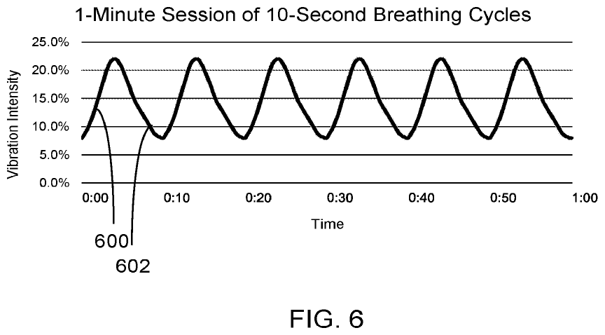

In one illustrative embodiment, a session of one minute duration that is made up of 10-second Breathing Cycles (for the average optimal breathing rate of 6 BPM) is shown in FIG. 6. Each cycle includes rising vibration intensity values 600 followed by falling vibration intensity values 602 for a total cycle duration of approximately 10 seconds. In general, each cycle should be between six and twelve seconds in duration.

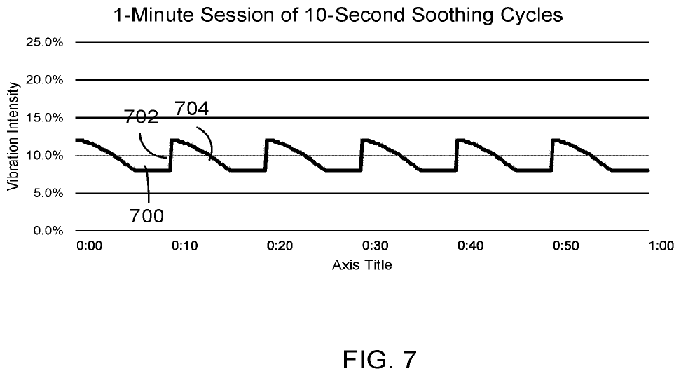

FIG. 7 illustrates a soothing session with a soothing rhythm to relax and release tension as vibration intensity values fall gradually. As shown in FIG. 7, each cycle includes consistent nominal vibration intensity values 700 followed by at least one (e.g., sharply) rising vibration intensity value 702 followed by (e.g., gradually) falling vibration intensity values 704 for a total cycle duration of approximately 10 seconds. In general, each cycle should be between six and twelve seconds in duration.

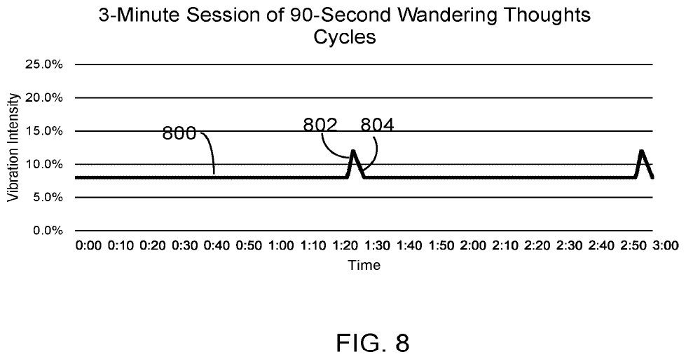

FIG. 8 illustrates a wandering thoughts session. This session involves subtle constant input and periodic pulses at intervals that would commonly be used by meditation instructors to help meditators become aware if their thoughts have wandered from the meditation's point of focus. That is, the periodic pulses nudge meditators to bring their thoughts and attention back into meditation. As shown in FIG. 8, each cycle includes consistent nominal vibration intensity values 800 followed by at least one rising vibration intensity value 802 followed by falling vibration intensity values 804. The consistent nominal vibration intensity values 800 are at least three-quarters of each cycle. In the example of FIG. 8, the consistent nominal vibration intensity values 800 occur over a duration of over one (1) minute and the rising vibration intensity value 802 followed by falling vibration intensity values 804 are approximately ten seconds in duration or less.

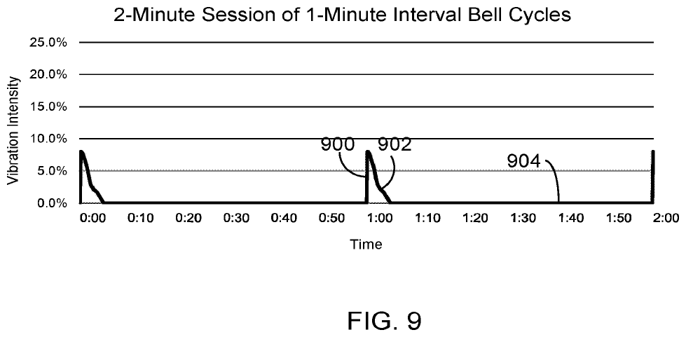

FIG. 9 illustrates an interval bells session. Each cycle has at least one rising vibration intensity value 900 followed by falling vibration intensity values 902, followed by an absence of vibration intensity values 904. In some implementations, the at least one rising vibration intensity value 900 followed by falling vibration intensity values 902 may comprise about a quarter (i.e., 25%) or less of each cycle. In the example of FIG. 9, the rising vibration intensity value 900 followed by falling vibration intensity values 902 comprise approximately five seconds in duration of a sixty-second cycle or one twelfth of a cycle.

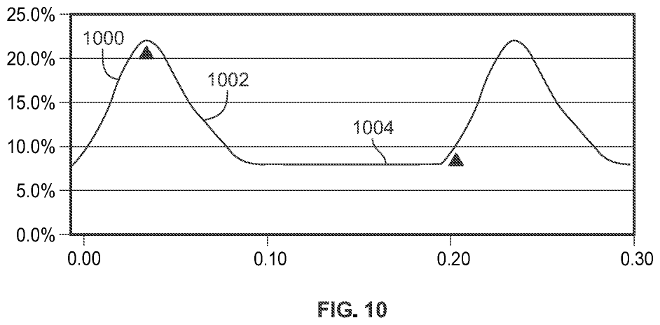

FIG. 10 illustrates an instructor session. This session may be used in conjunction with a live or recorded instructor to facilitate rhythmic counting, controlled breathing exercises, and physical awareness exercises. Each cycle may include rising vibration intensity values 1000 followed by falling vibration intensity values 1002 followed by consistent nominal vibration 1004 intensity values. The rising vibration intensity values 1000 followed by the falling vibration intensity values 1002 have a combined duration approximately equal to the duration of the consistent nominal vibration intensity values 1004.

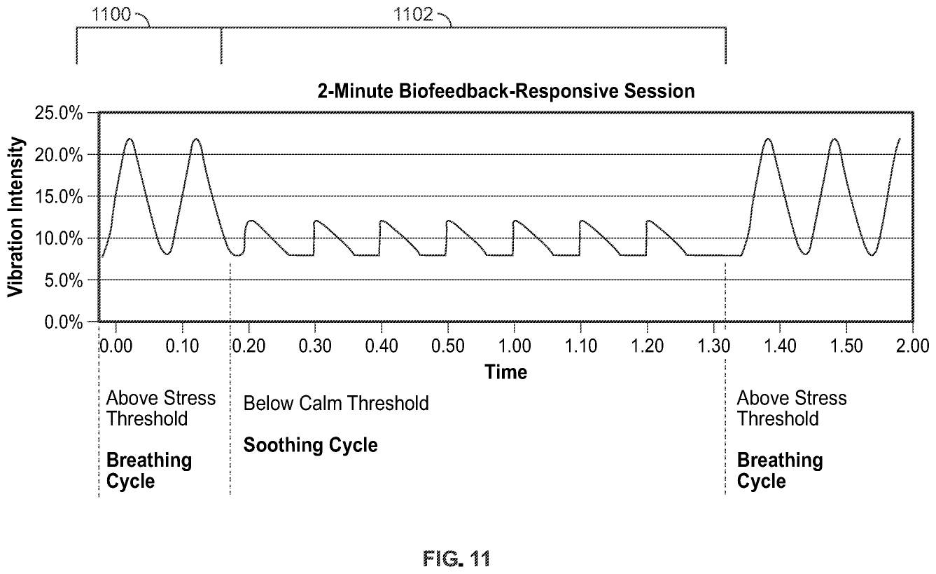

FIG. 11 illustrates an illustrative biofeedback session. In some implementations, a user's biofeedback may be used to dynamically alter the sequence of cycles for a session. For example, biometric thresholds may be set that represent stress and calm states and different cycles may be chosen for each state. Any cycle can be used for any given biometric state. The choice of cycle for each state is made either by pre-set defaults, analyzing previous sessions, and other users' data to select cycle sequences that have been most effective for the specific user (or for users like her), user choice, or instructor choice.

The exemplary session of FIG. 11 uses the breathing cycle (FIG. 6) when a user is above a (e.g., pre-determined or desired) biometric stress threshold, in order to help calm down the user. Upon achieving a calm biometric threshold, the vibration fades into a soothing cycle (FIG. 7). If the user returns to the stressed state, the breathing cycle starts again. That is, FIG. 11 illustrates a breathing session segment 1100, followed by a soothing session segment 1102. During the breathing session segment 1100 each cycle includes rising vibration intensity values followed by falling vibration intensity values, as shown in FIG. 6. During the soothing session segment 1102 each cycle includes consistent nominal vibration intensity values followed by at least one rising vibration intensity value followed by falling vibration intensity values, as shown in FIG. 7. The biofeedback session includes a sequence of breathing session segments 1100 followed by a sequence of soothing session segments 1102. In some implementations, the sequence of soothing session segments 1102 may be at least twice as long as the sequence of breathing session segments 1100.

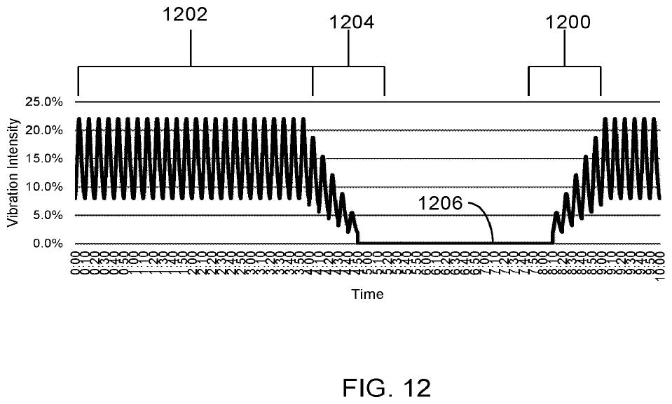

FIG. 12 illustrates a fade session. For any of the sessions described here, an optional fade may be applied. This means that approximately 40% through a timed meditation, or after a set number of minutes, the vibration cycles slowly fade out, as the user is deep in her meditation flow. The cycles slowly fades back in at approximately 85% through the meditation or after a set number of minutes. This allows the user to gently become aware of her physical surroundings and sensations as she ends a meditation session—a tangible version of instructor guidance towards the end of a session to become aware of physical feelings, sounds, and physical surroundings before opening one's eyes and completing a meditation.

As shown in FIG. 12, the fade session typically includes a ramp-up session segment 1200 with a first sequence of cycles in which each cycle in the first sequence of cycles has rising vibration intensity values followed by falling vibration intensity values that are greater than rising vibration intensity values and falling vibration intensity values of a previous cycle. Once the vibration intensity values reach a maximum vibration intensity value, a steady-state session segment 1202 having substantially identical rising vibration intensity values followed by substantially identical falling vibration intensity values begins. Subsequently, a ramp-down session segment 1204 having a second sequence of cycles in which each cycle in the second sequence of cycles has rising vibration intensity values followed by falling vibration intensity values that are greater than rising vibration intensity values and falling vibration intensity values of a subsequent cycle begins. A quiescent session segment 1206 may omit vibration intensity values altogether. In an illustrative implementation, the ramp-up session segment 1200 comprises less than a quarter (i.e., <25%) of the fade session duration, the steady state session segment 1202 comprises at least one half (i.e., ≥50%) of the fade session duration, the ramp down session segment 1204 is less than a quarter (i.e., <25%) of the fade session segment, and the quiescent session segment 1206 is less than a quarter (i.e., <25%) of the fade session duration.

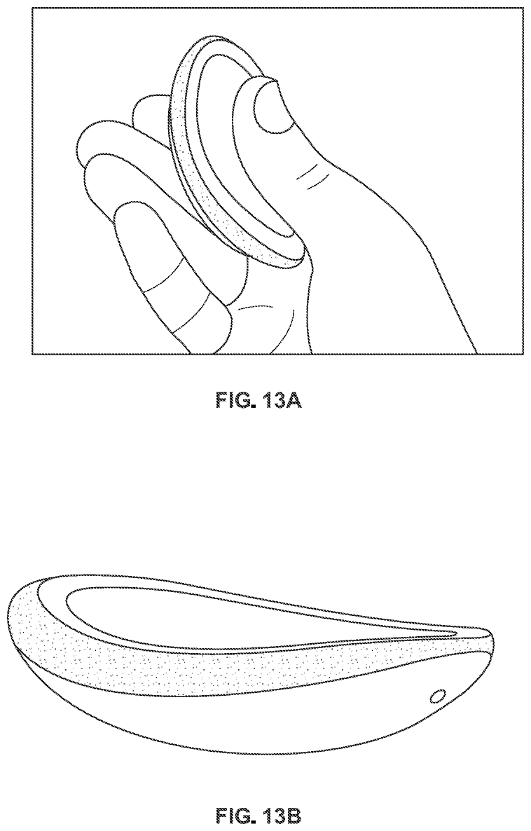

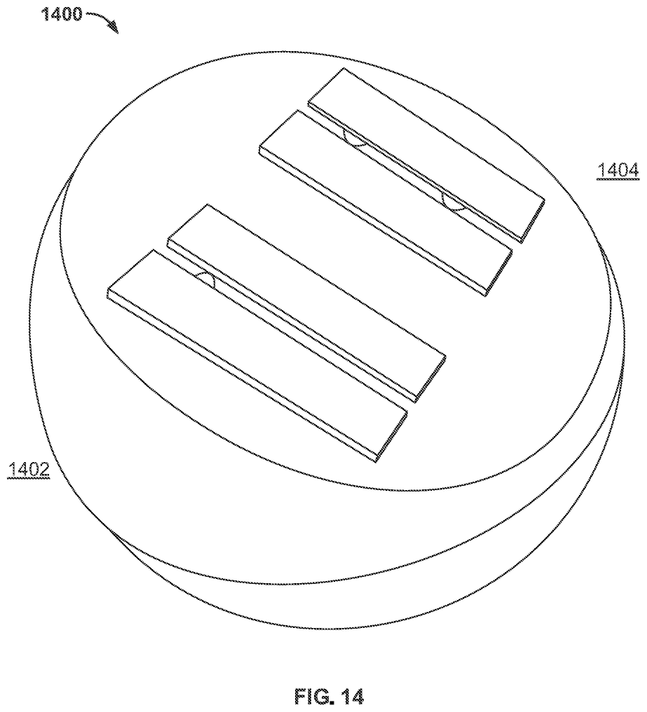

In general, the HSD 108 can have any suitable shape. For example, FIGS. 13A and 13B illustrate an HSD 108 having a shape that differs from that shown in FIGS. 3-5. FIG. 14 illustrates an HSD 108 having a pair of dry electrodes 1402, 1404 disposed in the concave portion of the HSD 108.

In various embodiments, the present invention can also include one or more of the following additional features:

Vibration Sound Dampening: In various embodiments, the system 100 may be adapted to feature sound dampening of the vibration motor 210 to achieve recued (e.g., near silent) vibration. For example, the vibration motor 210 can be clamped vertically, for example, with foam padding. A custom molded foam housing can be located precisely around the motor 210 itself. In some cases, the chamber that houses the motor 210 may be sized so as to be as small as possible to reduce reverberation.

Core Sport: In some embodiments, certain design modifications may be made such that the HSD 108 has a more “sport” design. These modifications would reduce the production cost and allow the product to be sold at a lower price point. The modifications would also involve removing the wood base and replacing it with plastic parts and a silicone overmold, removing the charging cradle and integrating USB-C charging into the main object, and other structural and material changes to allow the device to be more easily taken on the go.

Core Stone: In various embodiments, the HSD 108 may be structured and arranged to have the design and shape as shown in FIGS. 13A and 13B. In some embodiments, the exemplary HSD 108 includes a rounded, convex bottom portion and a concave top portion and may be adapted to be held in only one hand instead of two hands. In some cases, the HSD 108 may be adapted to have no or, optionally, limited biofeedback; however, in some cases, may be adapted to have a haptic pattern synced with mobile application content.

Single application controls multiple devices via Bluetooth: In various embodiments, a single mobile application can control multiple devices (e.g., up to 2, 3, 4, 5, 10, 15, 20, or 50 devices), e.g., via Bluetooth. For example, the application can detect nearby compatible devices. The application and firmware on the device may be used together to guide seamless selective Bluetooth pairings with the device(s), pairing devices with which the user wants to pair and excluding devices with which the user does not want to pair. In some instances, the application enables features and content depending on the type of device it connects with.

Core Unplugged: In various embodiments, a memory inside the device can record completed sessions and metadata, including, for example: duration, location (e.g., via GPS of device or phone), session type, and biofeedback (if applicable for that device). The device can determine when a user is explicitly starting a session by detecting the users' contact with the electrodes for a certain period of time. When in range of the application, the system may upload the data to the application and software may determine how the uploaded sessions integrate into history, personal biofeedback baselines, and usage-based insights. Advantageously, firmware provided on the device may be configured to determine in what order to replace or erase session data once uploaded, or in the event that the memory is full on the device.

Multiple vibration motors: In various embodiments, the device may be adapted to include multiple vibration motors on different axes such that each motor can be used independently or, in an array to create vibration patterns that a single vibration motor could not. An example is at least 3 Linear Resonant Actuators (LRAs) pointed in different direction which work in sync in order to create the vibration pattern of water swirling in a bowl.

Heating elements for data improvement: In various embodiments, the system may be configured to include a heating element to improve accuracy of heartrate and ECG data. Cold hands and circulation issues (e.g. Reynaud's syndrome) may interfere with precise readings via electrodes; thus, a heating element include in the device may be structured and arranged to improve circulation at the point of hand/finger contact can improve data readings in these scenarios.

Thermal elements for soothing: In various embodiments, the system may be adapted to include a temperature control element to provide hot and cold soothing effects for sessions, e.g., felt in the palm of the user's hands.

Live biofeedback via lights: In various embodiments, the system may be structured and arranged to use LEDs to display live biometric data readings. In general any measurable parameter can be displayed, e.g., heartrate and HRV readings.

Air inflation/deflation: In various embodiments, the HSD 108 may be structured and arranged to include a soft-sided form factor capable of inflating and deflating. In use, inflation/deflation of the HSD 108 may be used as a technique guide of user breathing patterns to mimic a body breathing.

A timeline graphic of a session. In various embodiments, the application may be adapted to display an interface with annotations for what exercises/guidance/techniques were being guided in the audio-haptic track, and show correlation to an individual user's biofeedback data during the same time windows, all via the same timeline graphic. In some variations, the software may be adapted to use metadata in the content management system (CMS) and biofeedback recorded during the session.

Gesture language with IMU: In various embodiments, the device may be structured and arranged to include an inertial measurement unit (IMU). In such instances, the device can be controlling using a series of gestures, e.g., where the user tips, taps, shakes, and rotates the device. In some embodiments, the user can control selecting a different session type, session start, session pause, and cycling through options by movements with the device instead of having to control these in the application.

Chunkable sessions based on biofeedback: In various embodiments, the system may be adapted to have the ability to dynamically create a session (comprised of shorter pre-recorded audio with matching/synchronized haptic patterns) in real-time. As one example, while a first portion of a session (e.g., Chunk 1) is playing, the system may be configured to detect heart rate and/or heart rate variability (HR/HRV) and then algorithmically select a second portion of the session (e.g., Chunk 2) based on the data. Once Chunk 1 has been completed, the system would play Chunk 2 seamlessly until the session ends. Although only two portions have been described, those of ordinary skill in the art can appreciate that a session may include more than two Chunks. Furthermore, the system may include a library of pre-recorded audio-haptic ‘chunks’ of content tagged with metadata for the algorithm, resulting in a near infinite combinations/Sessions. This feature can include: (i) combining multiple pre-recorded audio- haptic portions (i.e., Chunks) into complete sessions for a near infinite number of sessions, created programmatically, and (ii) doing that dynamically based on real-time data, which is to say, building the session while the session is running.

Haptic auto-authoring system: In various embodiments, the system may be structured and arranged to take an input of any music file and creates a haptic pattern that syncs with the music and creates a unique pattern of haptics (e.g., within certain guidelines of the system), so the user feels like it smoothly matches the music.

Respiration rate detection via bioimpedance: In various embodiments, the system may be structured and arranged to detect a user's respiration rate using bioimpedance. Bioimpedance measurements require passing a mild current (e.g., about 16 μA) through an individual's body. For example, current may be introduced (e.g., using a driving electrode) at a first, driving location and captured (e.g., using a sensing electrode) at a second, sensing location. Conventional respiration tracking using bioimpedance, however, has been performed by placing the driving and sensing devices on the individual's chest, proximate the individual's lungs, where the expansion and contraction of the individual's lungs during respiration create a discernible change in impedance to current flow. Conventional tracking also required use of a highly conductive gel to reduce contact impedance between the electrodes and the skin. Typical drive frequencies for the current may range between about 8 kHz and 32 kHz.

Thumbs are the extremities of the upper body, making it complicated to capture the change in bioimpedance in the thorax region during respiration. Problematically, historically, remote respiration tracking with the drive and sense electrodes placed on individuals' thumbs was not practical. Gel electrodes have been proposed but they are sticky and are not comfortable to apply and remove from thumbs; hence, a feasible option for remote tracking of respiration is to go for dry electrodes.

Efforts to implement remote respiration rate detection using dry electrodes are complicated by the fact that the contact impedance introduced via dry electrodes may be measured in the mega-ohm range. To overcome the contact impedance problem, the respiration rate measuring device may include a pair of driving electrodes and a pair of sensing electrodes in tetra-polar configuration. For example, referring to FIG. 14, an HSD 1400 that can measure a user's respiration rate remotely by measuring the patient's respiration from the patient's thumbs is shown. In some embodiments, the HSD 1400 includes a first pair of drive and sense (e.g., dry) electrodes 1402 and a second pair of drive and sense (e.g., dry) electrodes 1404. In operation, the drive and sensing electrodes on each thumb are adapted to cancel out the contact impedance, enabling capture of the change in impedance due to the expansion and contraction of the user's lungs. Although an HSD 1400 structured and arranged to measure the patient's respiration via the patient's thumbs is shown and described, those skilled in the art can appreciate that the device 1400 can be configured to contact any suitable anatomical region.

Applicant also discovered acceptable results can be obtained if current is delivered using non-conventional amperage and at non-conventional drive frequencies. For example, Applicant discovered that delivery of current at amperes higher than the conventional 16 μA, e.g., in a range from 18-60 μA, 22-52 μA, 26-44 μA, or 30-36 μA (e.g., 28 μA, 29 μA, 30 μA, 31 μA, 32 μA, 33 μA, 34 μA, 35 μA, or 36 μA); and/or at higher drive frequencies than the conventional 8-32 kHz, e.g., 36-128 kHz (e.g., 60 kHz, 70 kHz, 80 kHz, 90 kHz, and/or 100 kHz). Applicant also recognized that if either or both of the current and drive frequency is too high it may cause unacceptable damage. Indeed, in some embodiments, use of tetra polar electrodes configuration in conjunction with the non-conventional amperage and/or drive frequencies may result in acceptable respiration rate measurements. In some cases, the electrodes can be dry (e.g., without a gel to reduce contact impedance).

Respiration rate detection via IMU: In various embodiments, the system may be structured and arranged to include an accelerometer and/or a gyroscope that are adapted to measure inhale and exhale and fidget movements during sessions.

Guided respiration sessions with respiration rate detection: In some implementations, guided respiration sessions along with quantitative feedback of the users' breathing patterns may be important tool in health and fitness. Exemplary areas of applications may include, for the purpose of illustration rather than limitation: live, guided meditation sessions in a meditation studio, online guided meditation sessions with a live instructor, guided respiration sessions to improve mental and physical health situations such as remote treatment for hyperventilation, and so forth.

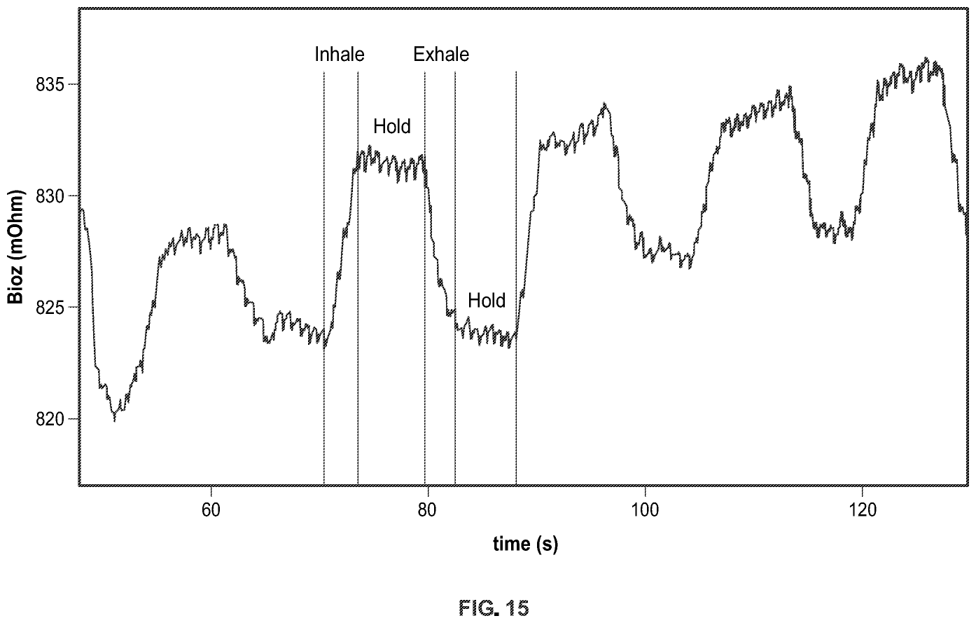

Referring to FIG. 15, an example of a waveform for bioimpedance measurements due to the expansion and contraction of the user's lungs during respiration over time are shown. Each cycle includes an inhale stage, during which there is an increase in bioimpedance due to the expanding lungs; a hold stage, during which the bioimpedance remains relatively unchanged; and an exhale stage, during which there is a decrease in bioimpedance due to the contracting lungs. Typically, the nature of the three stages vary based on the application needs, as well as the user.

In a group setting involving a plurality of users, the individual leading the respiration/breathing training (hereinafter, the “instructor”) uses her own breathing pattern to train the users to replicate that pattern, for example, in a closed loop manner. In some implementations, this may be accomplished by generating tactile and/or visual feedback that prompt each user to match the waveform of the instructor's breathing patterns.

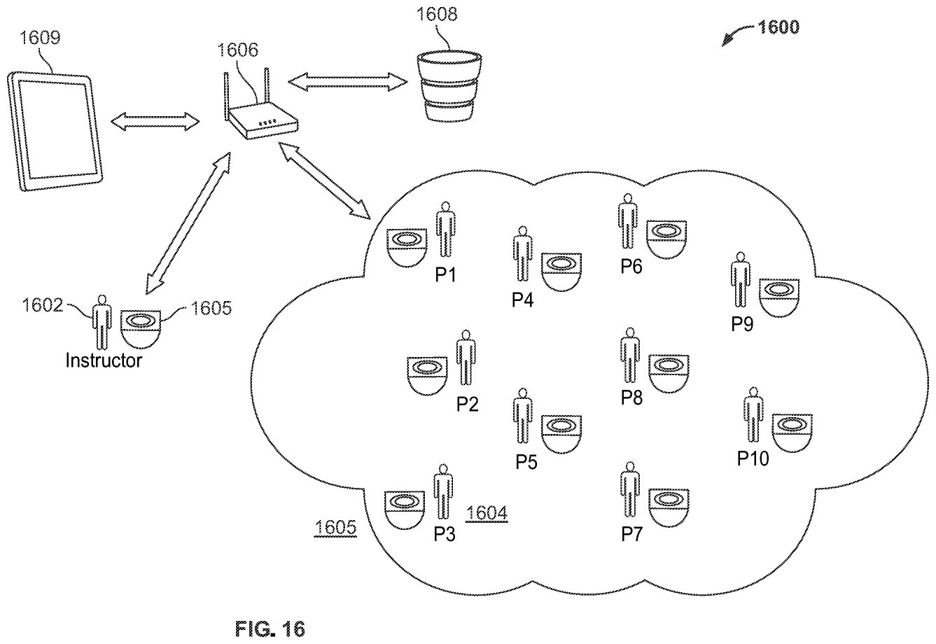

FIG. 16 shows an illustrative studio/room setting 1600 containing an instructor 1602 and a plurality of users 1604. The instructor 1602 and each of the users 1604 may each be equipped with an HSD 1605 that is in communication (e.g., via a localized, wireless communication network) with a processing device (e.g., a base station 1606) that, in some variations, may include or be in communication with a memory 1608 for storing data and an input/output device 1609. In operation, the instructor's breathing pattern may be captured on the instructor's HSD 1605 and transferred to the base station 1606. An algorithm, driver program, software, and the like stored on the base station 1606 or in memory 1608 may be adapted to communicate the instructor's breathing pattern to the HSDs 1605 of each of the users.

The users' HSDs 1605, in turn, may be structured and arranged to compare the instructor's breathing pattern with the breathing pattern of the individual user 1604 in real-time and guide the users to follow the instructor. Advantageously, the HSDs 1605 may be adapted so that discrepancies between the breathing patterns trigger tactile and/or visual feedback to guide each user 1604 to (e.g., precisely) match the inhale, hold, and exhale stages of the instructor's breathing pattern. Advantageously, the HSDs 1605 may also be adapted to transfer data concerning the user's breathing performance, adherence to the breathing protocols, heart rate, and so forth with the base station 1606. Optionally, these data may be displayed on the input/output device 1609 (e.g., a display screen) and/or stored in memory 1608.

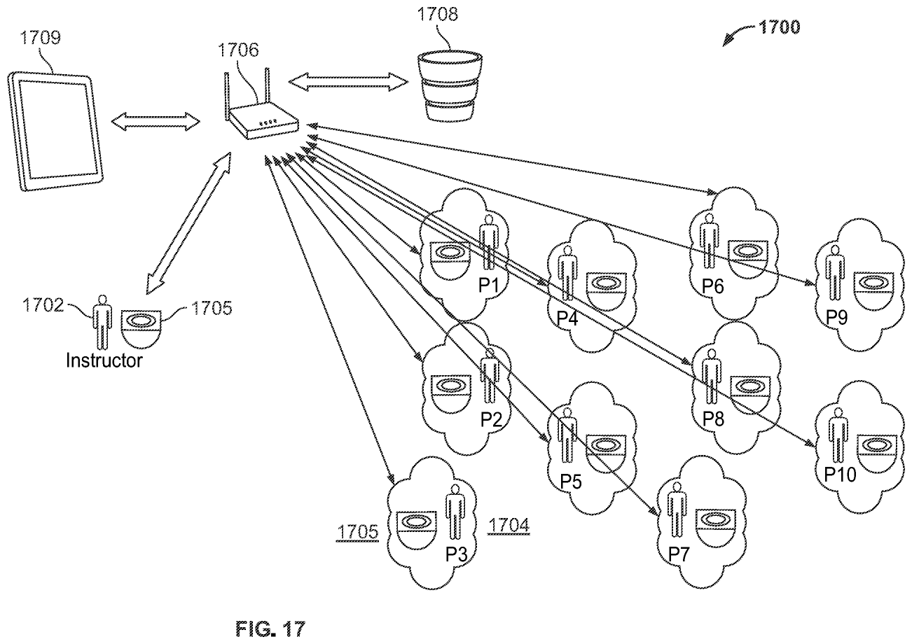

FIG. 17 shows an illustrative remote setting 1700 in which the instructor 1702 and the plurality of users 1704 are located remotely from one another. The instructor 1702 and each of the users 1704 may be equipped with an HSD 1705 that is in communication (e.g., via a wireless communication network) with a processing device (e.g., a base station 1706) that, in some variations, may include or be in communication with a memory 1708 for storing data and an input/output device 1709. In operation, the instructor's breathing pattern may be captured on the instructor's HSD 1705 and transferred to the base station 1706. An algorithm, driver program, software, and the like stored on the base station 1706 or in memory 1708 may be adapted to communicate the instructor's breathing pattern with the HSDs 1705 of each of the users 1704.

The users' HSDs 1705 compare the instructor's breathing pattern with the breathing pattern of the individual user 1704 in real-time. Advantageously, the HSDs 1705 may be adapted so that discrepancies between the breathing patterns trigger tactile and/or visual feedback to guide each user 1704 to (e.g., precisely) match the inhale, hold, and exhale stages of the instructor's breathing pattern. Advantageously, the HSDs 1705 may also be adapted to transfer data concerning the user's breathing performance, adherence to the breathing protocols, heart rate, and so forth with the base station 1706. Optionally, these data may be stored in memory 1708.

Haptics that respond to biofeedback in real time: In various embodiments, the system may be structured and arranged to alter or change the haptic patterns (e.g., strength, duration, and cadence) during a session based on real-time biofeedback. For example, in some instances, on rolling time windows, the software may be adapted to analyze the ECG/HRV/respiration biofeedback data from the user, compare these data to internal models and patterns, and determine if the user would be more successful if the haptics were different (e.g., slower, faster, stronger, weaker). In various embodiments, haptic patterns haptic patterns may be used to guide mental exercises, e.g., body scans, counting breath, mantras, and other mindfulness-based mental exercises.

On-device lighting as cue/reminder: In various embodiments, the device's LEDs may be configured to light up based on certain parameters. For example, user-set reminder schedule, recommended or predictive reminder schedule, detection of when user is in physical range of the device, and the like.

On-Device Social Reminders: In various embodiments, the device's lighting can light up when a “friend” (e.g., specified digitally in the application) performs or completes a set of actions, e.g., completes sessions or sends a notification (“nudge”).

AI/ML Interactive Reflection experience: In various embodiments, the system may be configured to use artificial intelligence/machine learning (AI/ML) algorithms to enhance functionality. Exemplary features using AI/ML may include, for the purpose of illustration rather than limitation: prompts, application suggest tags, and questions for the user's post-session journal entry and session annotations. Advantageously, the suggestions may be based on past activity, prior biometric data, biometrics from a most recent session, and/or data from populations similar to the user (e.g., beginnings of mental state classification).

Personalized Efficacy Algorithms: In various embodiments, the system may be structured and arranged to use the history of HRV-based results for a user to determine which mind & breathing techniques are most effective for specific goals (e.g., user-specified or system- specified goals). In some instances, system-specified goals may include, for example, biometric improvements, activity-retention, and/or desired patterns of engagement.

Population Efficacy Algorithms: In various embodiments, the system may be configured to use anonymized HRV-based results of groups of users to classify effects of different mind & breathing techniques and sequences of techniques on different population classifications.

Person classification in population: In various embodiments, the system may be adapted to use initial data about a single user to classify them within a population for the purpose of putting them on a recommendation path that is most likely to result in similar effects as the population group. In some instances, the recommendation path may include one or more of sessions, frequency, reminders, and/or other configurations in the mobile application. In some cases, the results may be targeted to include both biometric effects over time and behavioral/usage patterns.

Multi-modality Routines: In various embodiments, the system may be structured and arranged to use data about device ownership and activity and/or to combine session types from various devices (e.g., Core, Venom, HypericeX, Normatec, all made available from Hyperice™) to guide the user through routines that use multiple different types of devices.

Mental State Classification: In various embodiments, the system may be configured to use ECG and other personal data to classify various mental states of the user. Exemplary mental states may include, for the purpose of illustration rather than limitation: worried/anxious, exhausted/depleted, depressive/despair, joyful/happy, content/satisfied, positive excitement, neutral, and so forth.

Mental State Interventions: In various embodiments, the system may be adapted to use mental state classifications to identify interventions that are most likely to alter a discrete user's mental state. For example, the system may be configured to identify an exercise or series of exercises that affects moving from one state to another (e.g., from an anxious state to a neutral state), e.g., based on population data.

Integrating other data sources: In various embodiments, the system may be structured and arranged to integrate several data sources that represent sleep, fitness activity, nutrition, pregnancy, chronic conditions along with the HRV-based data to inform the interpretation of HRV, and to provide insights and triggers for a recovery intervention (an intervention could use any product/technique). In various instances, users may have the ability to authorize access to different third party services. For example, when granted access, the servers may be adapted to pull data from these other services and utilize them as inputs to the meditation recommendations algorithms, routine recommendations algorithms, personalized plans, HRV baseline calculations, heart rate typical ranges, classification of calm and focus, and personalized push and in-app notifications, and so forth.

ECG as unique user identifier: In various embodiments, the system may be structured and arranged to use an ECG as a thumbprint, e.g., to identify/differentiate one user from another automatically.

EDA and GSR sensing: In various embodiments, the system may be adapted to incorporate Electrodermal (EDA)/Galvanic Skin Response (GSR) sensing and skin temperature as additional signals to use in detecting stress levels and emotional state of a user.

An embodiment of the present invention relates to a computer storage product with a computer readable storage medium having computer code thereon for performing various computer-implemented operations. The media and computer code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs, DVDs and holographic devices; magneto-optical media; and hardware devices that are specially configured to store and execute program code, such as application-specific integrated circuits (“ASICs”), programmable logic devices (“PLDs”) and ROM and RAM devices. Examples of computer code include machine code, such as produced by a compiler, and files containing higher-level code that are executed by a computer using an interpreter. For example, an embodiment of the invention may be implemented using JAVA®, C++, or other object oriented programming language and development tools. Another embodiment of the invention may be implemented in hardwired circuitry in place of, or in combination with, machine-executable software instructions.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. The disclosed embodiments are contemplated in various combinations and permutations. It is intended that the following claims and their equivalents define the scope of the invention.

What is claimed is:

1. A hand stimulation device comprising:
a housing defining an interior plenum and comprising a top portion and a bottom portion;
a pair of drive electrodes structured and arranged in a first region of the top portion;
a pair of sense electrodes structured and arranged in a second region of the top portion;
a processing device disposed within the interior plenum and in communication with each of the pair of drive electrodes and the pair of sense electrodes;
at least one sensing device adapted to measure and collect biometric data about the user; and
at least one motor that is adapted to generate at least one of tactile feedback and a haptic pattern to a user.

2. The hand stimulation device of claim 1, wherein the pair of drive electrodes and the pair of sense electrodes are structured and arranged to measure a user's respiration rate remotely using bioimpedance.

3. The hand stimulation device of claim 2, wherein bioimpedance measurement comprises a first electrode of the pair of drive electrodes that is structured and arranged to deliver a current to a first electrode of the pair of sense electrodes in excess of 16 μA at a drive frequencies greater than 32 kHz.

4. The hand stimulation device of claim 3, wherein the first electrode of the pair of drive electrodes is structured and arranged to deliver current selected from the group consisting of: 28 μA, 29 μA, 30 μA, 31 μA, 32 μA, 33 μA, 34 μA, 35 μA, or 36 μA, a range from 18-60 μA, a range between 22-52 μA, a range between 26-44 μA, and a range between 30-36 μA.

5. The hand stimulation device of claim 3, wherein the first electrode of the pair of drive electrodes is structured and arranged to deliver current at a frequency selected from the group consisting of: 60 kHz, 70 kHz, 80 kHz, 90 kHz, 100 kHz, and a range from 36-128 kHz.

6. The hand stimulation device of claim 1, wherein each of the first pair of drive electrodes and the second pair of sense electrodes comprise dry electrodes.

7. The hand stimulation device of claim 1, wherein each of the first pair of drive electrodes and the second pair of sense electrodes comprises tetra polar electrodes.

8. The hand stimulation device of claim 1, wherein each of the first pair of drive electrodes and the second pair of sense electrodes is (i) configured to contact an anatomical region of the user remote from a chest of the user and (ii) adapted to capture a change in impedance due to an expansion (inhale) and a contraction (exhale) of the user's lungs.

9. The hand stimulation device of claim 8, wherein the anatomical region of the user comprises the thumbs of the user.

10. The hand stimulation device of claim 1, wherein the sensing device is selected from the group consisting of a heart rate sensor, a heart rate variability sensor, a moisture sensor, and a temperature sensor, an electrocardiogram (ECG), and combinations thereof.

11. The hand stimulation device of claim 1, wherein the sensing device is structured and arranged to transfer sensed data to at least one of the processing device or remotely.

12. The hand stimulation device of claim 11, wherein the sensed data are selected from the group consisting of a user's breathing performance, a user's adherence to breathing protocols, and a user's heart rate.

13. The hand stimulation device of claim 11, wherein the sensed data are selectively displayable on an input/output device.

14. The hand stimulation device of claim 1 further comprising a memory that is configured to include a plurality of programs for performing at least one of a plurality of hand stimulation sessions on the device.

15. The hand stimulation device of claim 14, wherein the memory is structured and arranged to record and store at least one session completed by the user.

16. The hand stimulation device of claim 1, wherein the at least one motor comprises a plurality of vibration motors configured an different axes.

17. The hand stimulation device of claim 1, further comprising a device for dampening sound generated by the at least one motor.

18. The hand stimulation device of claim 1 further comprising a heating element.

19. The hand stimulation device of claim 1 further comprising a plurality of light emitting devices that are adapted to display biometric data readings.

20. The hand stimulation device of claim 1, further comprising a form factor capable of being selectively inflated and deflated.

21. The hand stimulation device of claim 1, wherein the haptic pattern comprises a pattern that syncs with music.

22. A system for performing a meditative, breathing session, the system comprising:
a hand stimulation device comprising:
a housing defining an interior plenum and comprising a top portion and a bottom portion;
a pair of drive electrodes structured and arranged in a first region of the top portion;
a pair of sense electrodes structured and arranged in a second region of the top portion;
a processing device disposed within the interior plenum and in communication with each of the pair of drive electrodes and the pair of sense electrodes;
at least one sensing device adapted to measure and collect biometric data about the user; and
at least one motor that is adapted to generate at least one of tactile feedback and a haptic pattern to a user;
a client device in communication with the hand stimulation device; and
a server in communication with the client device and the hand stimulation device.
